(19) United States
(12) Patent Application Publication
(10) Pub. No.: US 2005/0187187 A1
(43) Pub. Date: Aug. 25, 2005

(54) Carboxyl-reduced derivatives of chondroitin sulfate, preparation thereof, use thereof as a medicinal product and the pharmaceutical compositions containing them

(75) Inventors: Wolfgang Ulmer, Eppstein (DE); Christian Viskov, Ris Orangis (FR); Philippe Hubert, Maisons-Alfort (FR)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharmas S.A., Antony (FR)

(21) Appl. No.: 11/016,175

(22) Filed: Dec. 17, 2004

Related U.S. Application Data
(60) Provisional application No. 60/618,662, filed on Oct. 12, 2004.

(30) Foreign Application Priority Data
Dec. 19, 2003 (FR) 0314988

(51) Int. Cl. A61K 31/737; C08B 37/00

(52) U.S. Cl. 514/54; 536/53

(57) ABSTRACT

The present invention relates to the carboxyl-reduced and chemoselectively O-sulfated derivatives of chondroitin sulfate of formula (I): 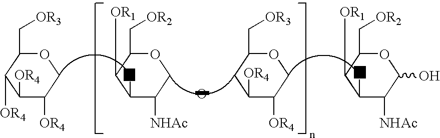
in which R₁, R₂, R₃ and R₄ are H or SO₃M, n is an integer of between 0 and 200, M is an alkali metal, isolated or as mixtures, to the diastereoisomers thereof, to the process for the preparation thereof, to the uses thereof as a medicinal product and to the pharmaceutical compositions containing them.

This application claims the benefit of U.S. Provisional Application No. 60/618,662 filed Oct. 12, 2004 and benefit of priority from French Patent Application No. 03 14988 filed Dec. 19, 2003, both of which are incorporated herein by reference in their entirety.

The present invention relates to novel glycosaminoglycans and also to their pharmaceutically acceptable addition salts, and more precisely to carboxyl-reduced and chemoselectively O-sulfated derivatives of chondroitin sulfate, isolated or as a mixture, to the use thereof as a medicinal product and to the pharmaceutical compositions containing them.

Glycoaminoglycans (GAGs) are essentially made up of alternating uronic acid-amino sugar (or vice versa) units of the type such as those encountered in the oligosaccharide or polysaccharide chains of biologically active natural GAGs such as heparin, heparan sulfate, dermatan sulfate, chondroitins, chondroitin sulfates or hyaluronic acid.

The uronic acid units correspond more specially to the D-glucoronic or L-iduronic acid structure and the amino sugar units to the D-galactosamine structure.

The natural GAGs exhibit therapeutic activities as thrombin inhibitors. They therefore exhibit antithrombotic and anticoagulant activity and are used in cardiovascular pathologies in which there is a risk of thrombosis.

Sulfated derivatives of chondroitin sulfate are described for their anticoagulant properties in patent application FR 2584728.

Moreover, O-persulfated derivatives of chondroitin sulfate have been described and studied for their properties in rheumatoid arthritis in international application WO 92/13541-A1.

The applicant has developed a novel process using a carboxyl-reduction step and a sulfatation (sulfating) step for obtaining novel chondroitin sulfate derivatives exhibiting advantageous properties for treating or preventing disorders which display an increased activity of at least one of the matrix metalloproteinases.

The carboxyl-reduced and chemoselectively O-sulfated derivatives of chondroitin sulfate of formula (I) have a very regular polymeric structure with degrees of purity of the order of 90%. Reproducible and unexpected biological properties result therefrom.

In the pathological condition of osteoarthritis, the degradation of aggrecan, the main proteoglycan of cartilage in the joints, represents a very early and crucial event. The pathological loss of aggrecan is caused by proteolytic cleavages in its interglobular domain. Amino acid sequence analyses of the proteoglycan metabolites isolated from the synovial fluid of patients suffering from joint damage, from osteoarthritis or from an inflammatory disorder of the joints, have shown that proteolytic cleavage exists between the amino acids Glu³⁷³ and Ala³⁷⁴ in the interglobular domain of human aggrecan (Lohmander, et al., Arthritis Rheum., 36: 1214-1222 (1993)). The proteolytic activity responsible for this cleavage is called “aggrecanase” and can be attributed to the metalloproteinase (MP) or matrix metalloproteinase (MMP) superfamily.

Zinc is an essential element in the catalytically active center of metalloproteinases. MMPs cleave collagen, laminin, proteoglycans, elastin or gelatin under physiological conditions. They therefore play an important role in bone tissue and connective tissue. A large number of different MMP inhibitors are known (see, for example, EP 0 606 046; WO 94/28889). However, the known MMP inhibitors frequently have a significant disadvantage. They lack specificity for any particular class of MMP. On the contrary, most MMP inhibitors simultaneously inhibit a plurality of MMPs.

Consequently, a need exists for MMP inhibitors that have more narrowly defined specificities in order to more effectively treat or prevent specific disorders.

A subject of the invention is most particularly the carboxyl-reduced and sulfated derivatives of chondroitin sulfate of formula (I): 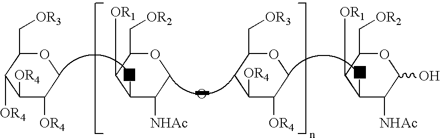
in which R₁, R₂, R₃ and R₄, which may be identical or different, represent H or SO₃M, it being understood that at least one of the substituents R₁, R₂, R₃ and R₄ represents a group SO₃M, n is an integer of between 0 and 200, M is an alkali metal, said derivatives being in the form of an isolated compound or in the form of mixtures, and also the diastereoisomers thereof.

In particular, a subject of the invention is the carboxyl-reduced derivatives of chondroitin sulfate according to formula (I), wherein M is chosen from sodium, calcium, magnesium and potassium, and in particular sodium.

Preferably, in formula (I), n is an integer of between 100 and 200.

A subject of the invention is more particularly the carboxyl-reduced derivatives of chondroitin sulfate as described above, wherein R₁, R₂, R₃ and R₄ represent SO₃Na.

A subject of the invention is more particularly the carboxyl-reduced derivatives of chondroitin sulfate as described above, wherein R₁, R₂ and R₄ represent SO₃Na and R₃ represents H.

A subject of the invention is more particularly the carboxyl-reduced derivatives of chondroitin sulfate as described above, wherein R₁ represents H or SO₃Na, R₂ represents SO₃Na and R₃ and R₄ represent H.

A subject of the invention is more particularly the carboxyl-reduced derivatives of chondroitin sulfate as described above, wherein R₁ represents H or SO₃Na, R₂ and R₃ represent SO₃Na and R₄ represents H.

These polysaccharides thus comprise an even number of saccharides.

In general, the mixtures of polysaccharides according to the invention can be prepared, firstly, by carboxyl-reduction of chondroitin sulfate or of its derivatives, followed by chemoselective sulfatation, or conversely by sulfatation followed by carboxyl-reduction.

A subject of the invention is therefore the process for preparing carboxyl-reduced and sulfated derivatives of chondroitin sulfate as defined above, using the following steps:
carboxyl-reduction of chondroitin sulfate
   either a) by means of a carbodiimide derivative (so as to form an activated adduct) and in the presence of a reducing agent,
   or b) by esterification, the chondroitin sulfate having, where appropriate, been trans-salified beforehand with a quaternary ammonium salt, followed by reduction of the corresponding intermediate ester derivative by the action of a reducing agent,
where appropriate, trans-salification (salt exchange) of the carboxyl-reduced derivative with a quaternary ammonium salt,
sulfatation in organic medium, followed by salification (salt formation).

The following reaction scheme illustrates the present invention:

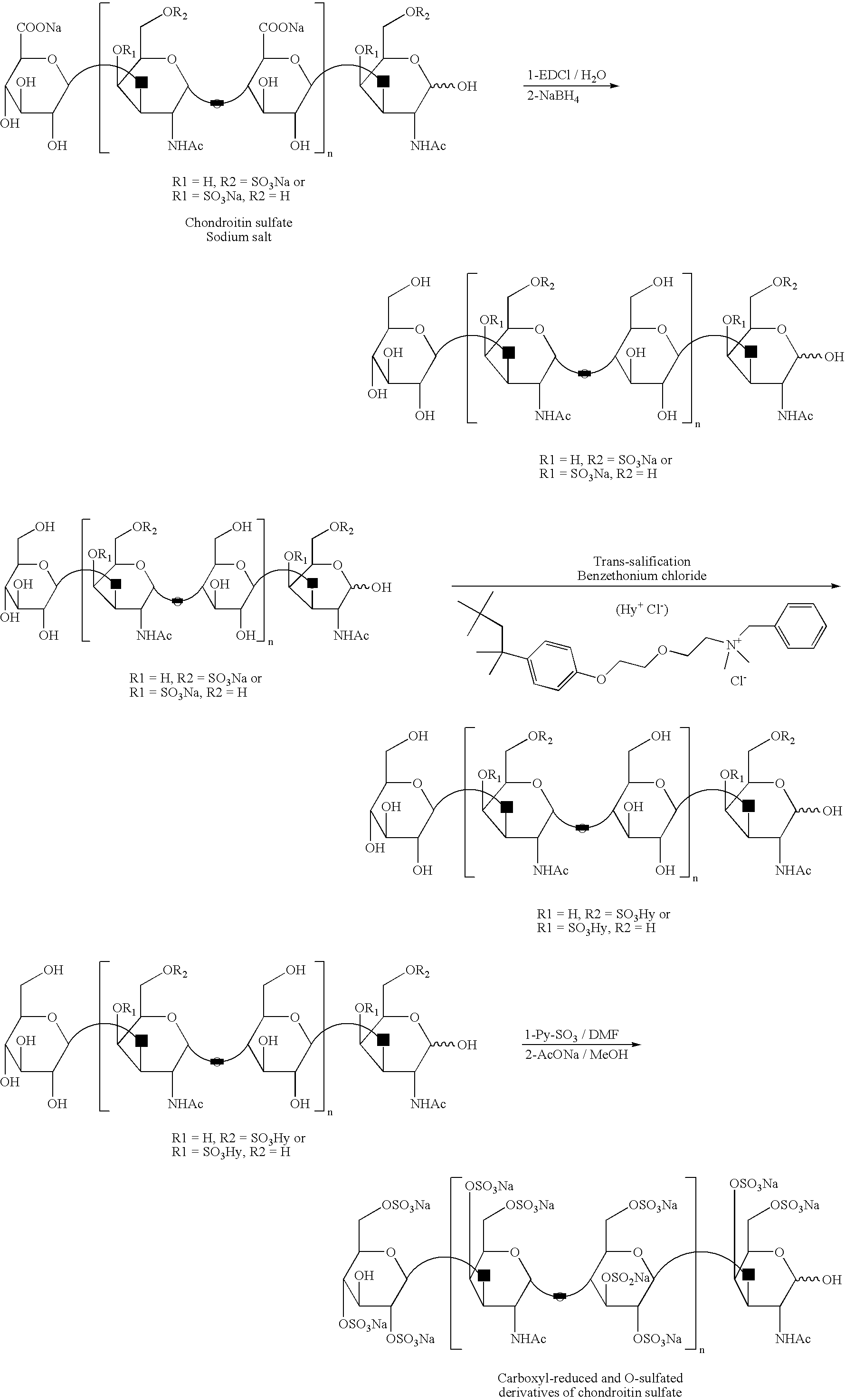

Alternatively, the carboxyl-reduced, chemoselectively O-sulfated derivatives of chondroitin sulfate can be obtained by sulfatation of a quaternary ammonium salt of chondroitin sulfate in organic medium by means of a complex of sulfuric anhydride with an organic base such as pyridine or trimethylamine, followed by carboxyl-reduction by means of a carbodiimide derivative such as 1-(3-dimethylaminopropyl)-3-ethylcarbodiimide hydrochloride, in the presence of a reducing agent such as sodium borohydride, or by means of an ester derivative, such as the methyl ester, in the presence of a reducing agent such as sodium borohydride.

The persulfated or 6-O-sulfated, carboxyl-reduced derivative of chondroitin sulfate can then be trans-salified with a quaternary ammonium salt and then optionally sulfated on the hydroxyl function in the 6′-position thus generated, by the action of a complex of sulfuric anhydride with an organic base such as pyridine or trimethylamine.

A subject of the invention is therefore also a process for preparing the carboxyl-reduced derivatives of chondroitin sulfate as defined above by successively carrying out the following steps:
trans-salification of chondroitin sulfate with a quaternary ammonium salt,
sulfatation in organic medium, followed by salification,
carboxyl reduction of the persulfated chondroitin sulfate
   either a) by means of a carbodiimide derivative (so as to form an activated adduct) in the presence of a reducing agent,
   or b) by esterification, the persulfated chondroitin sulfate having, where appropriate, been trans-salified beforehand with a quaternary ammonium salt, and then reduction of the corresponding intermediate ester derivative by the action of a reducing agent,
where appropriate, trans-salification of the persulfated, carboxyl-reduced derivative with a quaternary ammonium salt, and then resulfatation followed by salification.

The following reaction scheme illustrates the present invention:

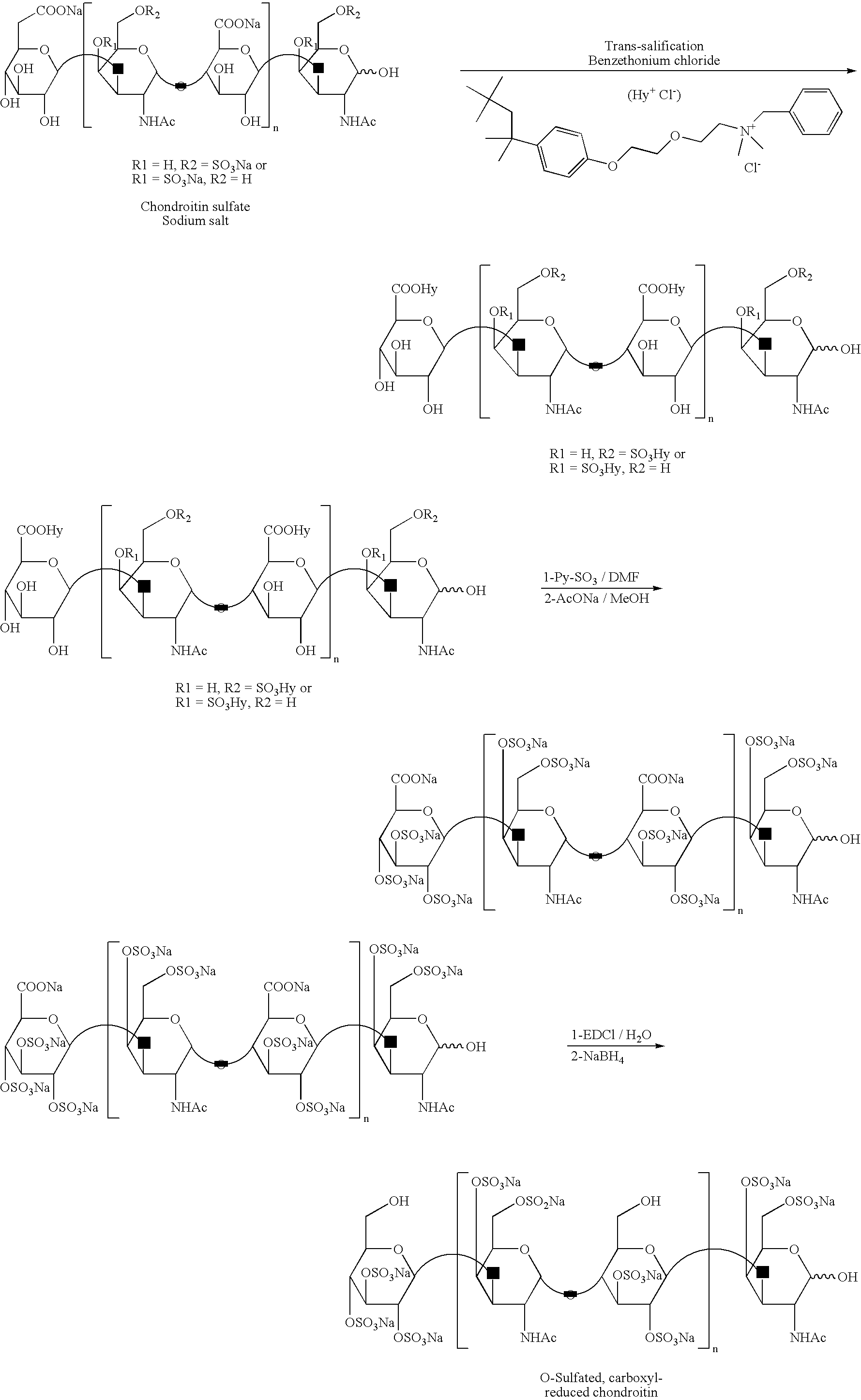

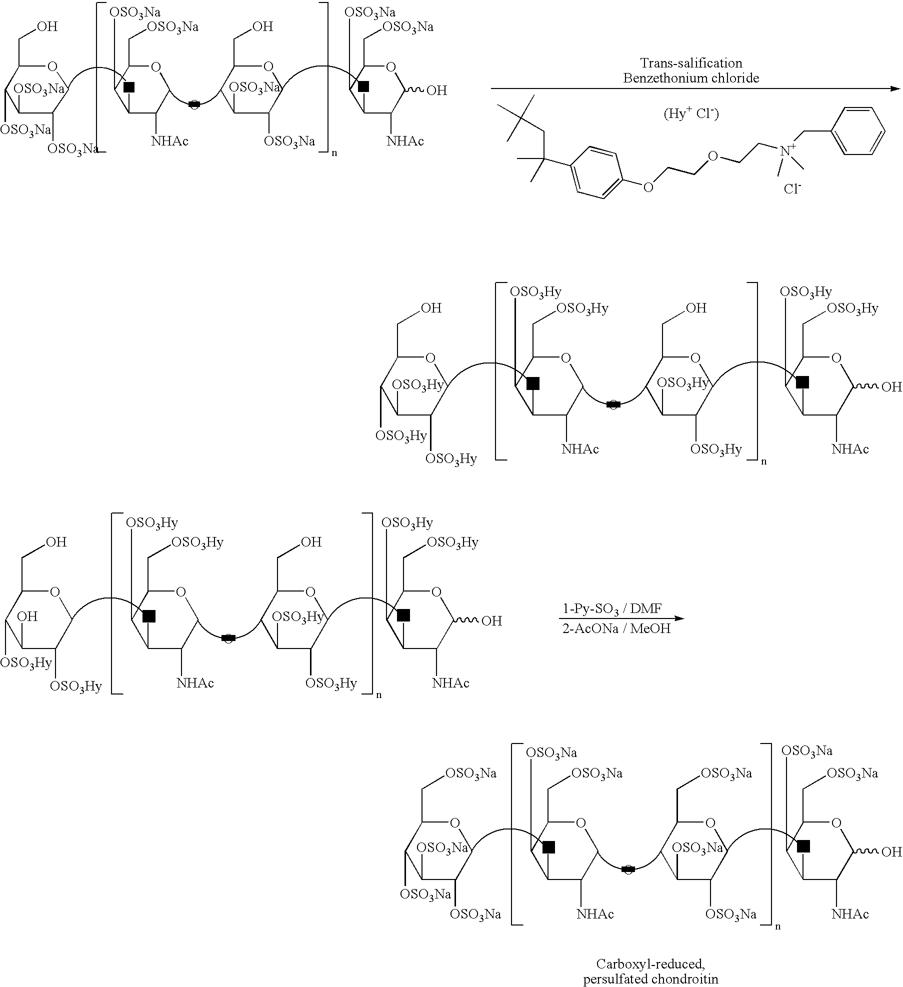

The processes according to the present invention are characterized by the strong chemoselectivity of the carboxyl-reduction and sulfatation reactions. The products which result therefrom are notably homogeneous and result in true polymers. The carboxyl-reduced chondroitin sulfate derivatives have purities of the order of 90%. This homogeneity is determined by NMR and infrared structural analysis.

Reproducible and unexpected biological properties result therefrom.

The persulfatation reactions are preferably carried out in organic medium by means of a complex of sulfuric anhydride with an organic base such as pyridine or trimethylamine. They are generally followed by a salification reaction, for example by the action of sodium acetate.

In general, the carboxyl-reduced or non-carboxyl-reduced derivative of chondroitin sulfate can be trans-salified with a quaternary ammonium salt and then chemoselectively O-sulfated on the hydroxyls in the 6- and 6′-positions only, or persulfated by reaction with a complex of sulfuric anhydride with an organic base such as pyridine or trimethylamine.

For optimal chemoselectivity of the reactions' for persulfatation of the carboxyl-reduced or non-carboxyl-reduced chondroitin sulfate, it is preferable to use a benzethonium salt in the presence of 10 to 30 equivalents of pyridine-sulfuric anhydride complex per hydroxyl function to be sulfated. For the same reason, the reactions should preferably be carried out at temperatures of between −15 and 70° C.

During the persulfatation of the benzethonium salt of the carboxyl-reduced chondroitin sulfate (compound of formula (I) with R₁═H/R₂═SO₃Na or R₁═SO₃Na/R₂═H and R₃═R₄═H), the procedure is preferably carried out at temperatures of between 50 and 70° C. This is illustrated in example 1.

For optimal chemoselectivity of the 6,6′-O-sulfatation of the carboxyl-reduced chondroitin sulfate benzethonium salt (compound of formula (I) with R₁═H/R₂═SO₃Na, R₁═SO₃Na/R₂═H and R₃═R₄═H), the procedure is carried out in the presence of 5 to 15 equivalents of pyridine-sulfuric anhydride complex, at temperatures of between −15 and 5° C. This is illustrated in example 2.

The persulfatation of the chondroitin sulfate benzethonium salt is, for its part, carried out at temperatures of between 50 and 70° C. This is illustrated in example 3.

Similarly, for optimal chemoselectivity of the 6-O-sulfatation of the chondroitin sulfate benzethonium salt, it is preferable to carry out the reaction in the presence of 5 to 15 equivalents of pyridine-sulfuric anhydride complex, at temperatures of between −15 and 5° C. Even more preferably, the reaction should be carried out in the presence of approximately 10 equivalents of pyridine-sulfuric anhydride complex, at a temperature in the region of 0° C. This is illustrated in example 4.

During the (re)sulfatation of the persulfated, carboxyl-reduced derivative of the chondroitin sulfate benzethonium salt (compound of formula (I) with R₁ ═R₂═R₄═SO₃Na and R₃═H), the procedure is preferably carried out in the presence of 10 to 30 equivalents of pyridine-sulfuric anhydride complex, at temperatures in the region of 20° C. This is illustrated in example 5.

For optimal chemoselectivity of the 6′-O-sulfatation of the 6-O-sulfated, carboxyl-reduced chondroitin sulfate benzethonium salt (compound of formula (I) with R₁═H/SO₃Na R₂═SO₃Na and R₃═R₄═H), it is preferable to carry out the reaction in the presence of 5 to 15 equivalents of pyridine-sulfuric anhydride complex, at temperatures of between −15 and 5° C. Even more preferably, the reaction should be carried out in the presence of approximately 10 equivalents of pyridine-sulfuric anhydride complex at a temperature in the region of −10° C. This is illustrated in example 6.

The carboxyl-reduced derivatives of chondroitin sulfate can be obtained by carboxyl-reduction of chondroitin sulfate or of its derivatives, according to the operating conditions described, for example, by R. L. Taylor and H. E. Conrad, Biochemistry, 11 (8), 1383-1388, (1972). The chondroitin sulfate is reacted with a soluble carbodiimide derivative in aqueous phase, such as 1-(3-dimethylaminopropyl)-3-ethylcarbodiimide hydrochloride, so as to form an activated adduct, which is reduced with a reducing agent such as sodium borohydride.

The term “derivatives (compounds) of chondroitin sulfate” is intended to mean in particular the chondroitin sulfate sodium salt, the chondroitin sulfate benzethonium salt, the sulfated derivatives of chondroitin sulfate, and also the sulfated derivatives of chondroitin sulfate benzethonium salts.

According to an alternative method, the carboxyl-reduced derivatives of chondroitin sulfate can also be obtained by carboxyl-reduction of an ester of chondroitin sulfate or of its chemoselectively O-sulfated derivatives.

By way of example, methyl esters of chondroitin sulfate or of its chemoselectively O-sulfated derivatives can be used and can be reduced by means of a reducing agent such as sodium borohydride.

The reaction with the carbodiimide derivative is preferably carried out in the presence of 5 to 20 equivalents of reactant and at a pH of between 4 and 5. Even more preferably, the reaction with the carbodiimide derivative is carried out in the presence of 7 to 13 equivalents of reactant and at a pH of between 4.3 and 4.9.

The reduction per se is carried out with a reducing agent known to those skilled in the art, and in particular with an alkali metal borohydride. By way of example, it is possible to use sodium borohydride.

The reduction per se of the activated adduct of the chondroitin sulfate derivative is carried out with 10 to 300 equivalents of alkali metal borohydride at a temperature of between 10 and 70° C. More preferably, the reduction of the abovementioned adduct is carried out in the presence of alkali metal borohydride at a temperature of between 10 and 30° C.

Most particularly, a subject of the invention is the process using the following steps:
either a) carboxyl-reduction of the chondroitin sulfate sodium salt by means of 1-(3-dimethyl-aminopropyl)-3-ethylcarbodiimide hydrochloride in the presence of sodium borohydride,
or b) esterification by the action of methyl iodide on the chondroitin sulfate derivative trans-salified beforehand with benzethonium chloride, and then reduction of the corresponding methyl ester derivative with sodium borohydride, then
trans-salification of the carboxyl-reduced derivative with benzethonium chloride,
sulfatation of the chondroitin sulfate benzethonium salt in organic medium by means of a complex of sulfuric anhydride with an organic base such as pyridine or trimethylamine, followed by salification with sodium acetate.

Alternatively, a subject of the invention is most particularly the process using the following steps:
trans-salification of the chondroitin sulfate sodium salt with benzethonium chloride,
sulfatation of the chondroitin sulfate benzethonium salt in organic medium by means of a complex of sulfuric anhydride with an organic base such as pyridine or trimethylamine, followed by salification with sodium acetate, then
either a) carboxyl-reduction of the chondroitin sulfate by means of 1-(3-dimethylaminopropyl)-3-ethylcarbodiimide hydrochloride in the presence of sodium borohydride,
or b) esterification by the action of methyl iodide on the chondroitin sulfate trans-salified beforehand with benzethonium chloride, then reduction of the corresponding methyl ester derivative with sodium borohydride,
then, where appropriate, trans-salification of the persulfated, carboxyl-reduced derivative with benzethonium chloride, then resulfatation with the sulfuric anhydride-organic base complex, followed by salification with sodium acetate.

When it is desired to obtain isolated derivatives from the mixture of carboxyl-reduced derivatives of chondroitin sulfate as obtained according to the process described above, the following process should then be applied to the mixture:
fractionation of the mixture by chromatography on columns filled with gel of polyacrylamide agarose type or a polyacrylamide gel. The mixture is eluted with a sodium hydrogen carbonate solution.

The sodium hydrogen carbonate solution is preferably a solution of 0.1 to 1 mol/l. Even more preferably, the separation is carried out at a concentration of 1 mol/l. The detection is carried out by refractometry.

A subject of the invention is also the carboxyl-reduced and chemoselectively O-sulfated derivatives of chondroitin sulfate, isolated or as a mixture as defined above, which can be obtained according to the process(es) as defined above.

The polysaccharides of formula (I), isolated or as mixtures, can be used as medicinal products.

These polysaccharides exhibit in particular strong inhibitory activity on certain matrix metalloproteases. These inhibitors are particularly indicated for the treatment of pathological states where a large increase in matrix metalloproteinase activity is noted.

The pathological states to which the present invention refers involve an increase in the activity of at least one of the following matrix metalloproteinases: neutrophil elastase, matrisilyn (MMP-7), aggrecanase, hADAMTS1 and gelatinase A (MMP-2).

The compounds can therefore be used for preventing and treating diseases such as degenerative joint disorders (such as osteoarthritis), spondylosis, chondrolysis associated with joint trauma or prolonged joint immobilizations (often following an injury to the meniscus or patella or the rupture of ligaments), disorders related to injuries (healing), periodontal disorders, chronic disorders of the locomotor system (such as chronic or acute inflammatory, immunological or metabolic forms of arthritis), arthropathies, or disorders related to bone metabolism.

A subject of the invention is also the pharmaceutical compositions containing the compounds of formula (I), isolated or as a mixture, and also one or more pharmaceutically acceptable excipients, vehicles or additives.

Another aspect of the invention is a process for preparing the pharmaceutical compositions containing the compounds of formula (I), wherein an amount corresponding to a desired dose of a compound of formula (I) is mixed with one or more pharmaceutically acceptable excipients, vehicles, additives.

The compounds of formula (I) can be administered via various routes. They may include, without being limited, subcutaneous, intraarticular, intraperitoneal or intravenous injections.

The administration may also be rectal, oral, by inhalation, or else transdermal.

In the case of solutions for injection (for example in the form of an ampoule), the doses may range from 5 μg to approximately 200 mg of compound of formula (I), and preferably from 10 μg to 40 mg.

The daily dose indicated for the treatment of an adult patient weighing approximately 70 kg is from 10 μg to 500 mg of active ingredients, generally from 20 mg to approximately 100 mg. However, depending on the circumstances, higher or lower daily doses may be appropriate.

These doses can be administered once a day in the form of a single dosage unit.

Alternatively, the doses can be administered in a plurality of smaller doses, given repeatedly at defined intervals over time.

The following examples illustrate the invention without, however, limiting it.

Chondroitin Sulfate

The chondroitin sulfate used for preparing the compounds illustrating this invention has a molecular mass of 85.2 kDalton. It is produced from fish cartilage and consists of a mixture of chondroitin A and chondroitin C in 50/50 proportions. Its chemical structure can be described by formula (I) with the groups R₁═SO₃M and R₂═R₃═R₄═H or R₁═H, R₂═SO₃M, R₃═R₄═H.

### EXAMPLE 1

### Preparation of the Carboxyl-Reduced and Persulfated Chondroitin Sulfate Sodium Salt (Compounds of Formula (I) with the Groups R₁═R₂═R₃═R₄═SO₃Na)

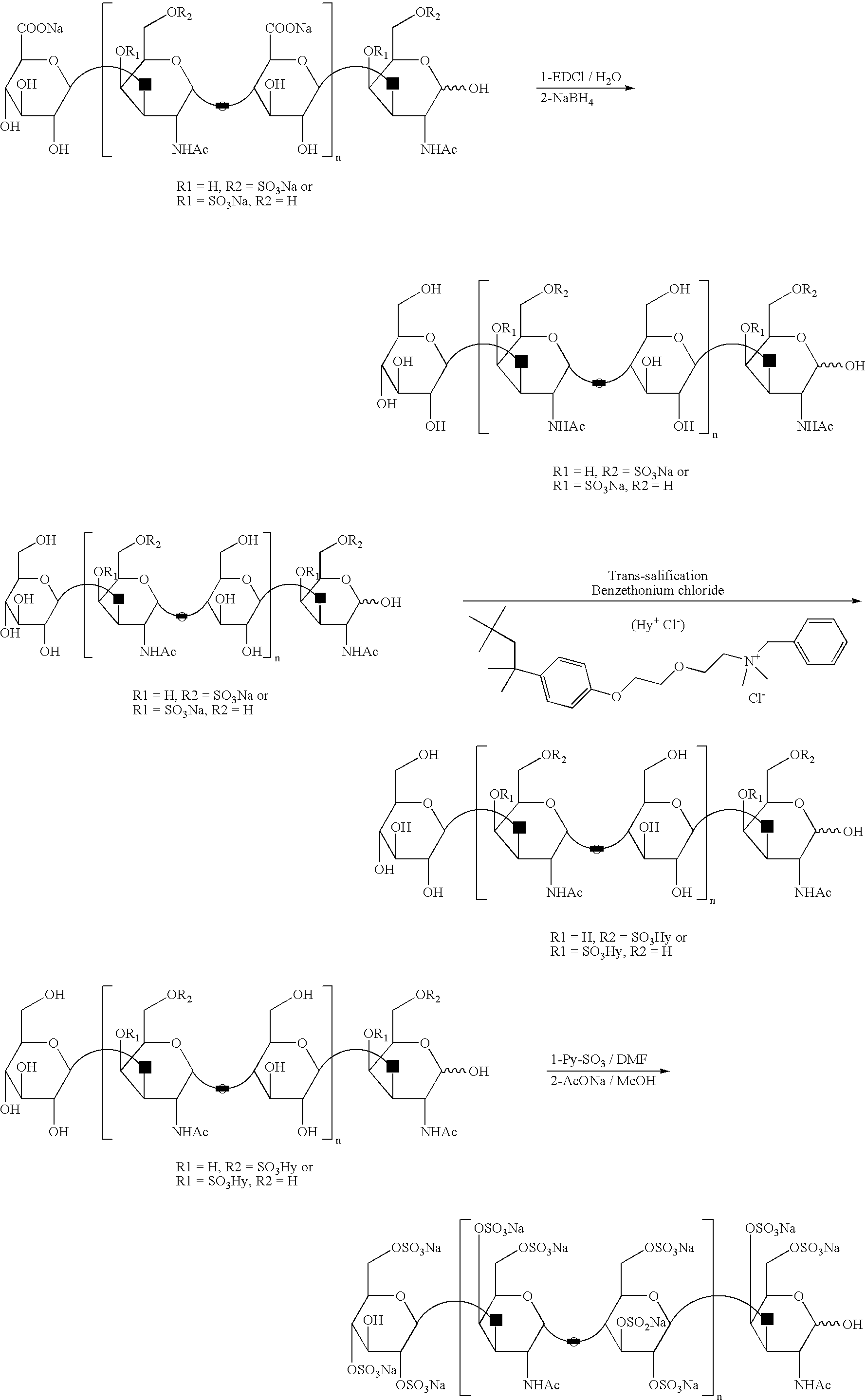

a) Preparation of the Carboxyl-Reduced Chondroitin Sulfate Sodium Salt

1 g of chondroitin sulfate sodium salt is dissolved, at a temperature in the region of 20° C., in 150 ml of water.

The pH of the solution is adjusted to a value of 4.7±0.1 by adding a 0.1 N hydrochloric acid solution. 3.45 g of 1-ethyl-3-(3-dimethylaminopropyl)carbodiimide hydrochloride are added and the mixture obtained is stirred while maintaining the pH at a value of 4.7±0.1 by adding a 0.1 N hydrochloric acid solution. When the pH is stabilized at a value in the region of 4.7, 1.8 g of sodium borohydride are added in small portions. The reaction medium is stirred for approximately 20 hours at a temperature in the region of 20° C. After cooling to a temperature in the region of 10° C., the reaction medium is neutralized by adding concentrated hydrochloric acid (10 N) to a pH in the region of 7. 600 ml of a 10% sodium acetate solution in methanol are added. The solid obtained is filtered, and then dissolved in 50 ml of water. 150 ml of a 10% sodium acetate solution in methanol are added to the solution obtained. The solid obtained is filtered, and then dissolved in 50 ml of water. The solution obtained is loaded into a PM 3500 cellulose ester membrane and dialyzed, changing the water baths regularly for 26 hours. The content of the membranes is lyophilized to give 0.682 g of carboxyl-reduced chondroitin sulfate sodium salt in the form of a white lyophilized material. The yield obtained is 73%.

Proton spectrum in D₂O, 400 MHz, T=333 K (δ in ppm—mixture of chondroitins A and C): 2.0 (3H, s), 3.30 (1H, m), between 3.4 and 3.65 (4H, m), between 3.7 and 4.05 (3H+1/2H, m), 4.18 (2H, s), between 4.4 and 4.6 (2H, m), 4.81 (1/2H, s)

b) Preparation of the Carboxyl-Reduced Chondroitin Sulfate Benzethonium Salt

A solution of 1.6 g of benzethonium chloride in 12 ml of water is added to the solution of 0.685 g of carboxyl-reduced chondroitin sulfate sodium salt in 10 ml of water. The suspension obtained is filtered. The cake is washed with water and then dried at 40° C. under reduced pressure (6 kPa) to give 1.25 g of carboxyl-reduced chondroitin sulfate benzethonium salt in the form of a cream solid. The yield obtained is 96%.

c) Preparation of the Carboxyl-Reduced and Persulfated Chondroitin Sulfate Sodium Salt

0.2 g of carboxyl-reduced chondroitin sulfate benzethonium salt is dissolved, at a temperature in the region of 60° C. and under an inert atmosphere, in 6 ml of anhydrous dimethylformamide. When dissolution is complete, a solution of 2.08 g of pyridine-sulfuric anhydride complex in 15 ml of anhydrous dimethylformamide is added to the above solution. After stirring for 1 hour 30 min at a temperature in the region of 60° C., the reaction medium is cooled to a temperature in the region of 10° C. 6 ml of water and then 0.90 ml of a 10% sodium acetate solution in methanol are added. The suspension obtained is filtered. The cake is dissolved in 30 ml of water and is then precipitated by adding 90 ml of a 10% sodium acetate solution in methanol. The suspension obtained is filtered. The cake is washed with methanol (twice 5 ml) and then dissolved in 50 ml of water. The solution obtained is loaded into a PM 3500 cellulose ester membrane and dialyzed, changing the water baths regularly for 30 hours. The content of the membrane is lyophilized to give 0.148 g of carboxyl-reduced and persulfated chondroitin sulfate sodium salt in the form of a white lyophilized material. The yield obtained is 74%.

Proton spectrum in D₂O, 400 MHz, T=298 K (δ in ppm—mixture of chondroitins A and C): 2.05 (3H, s), 3.85 (1H, m), between 3.92 and 4.05 (3H, m), between 4.12 and 4.3 (5H, m), 4.58 (1H, s), 4.65 (1H, s), 4.75 (1H, m), 4.95 (1H, s), 5.0 (1H, s).

### EXAMPLE 2

### Preparation of the Carboxyl-Reduced and 6,6′-O-sulfated Chondroitin Sulfate Sodium Salt (Compounds of Formula (I) with the Groups R₁═H/SO₃Na R₂═R₃═SO₃Na and R₄═H)

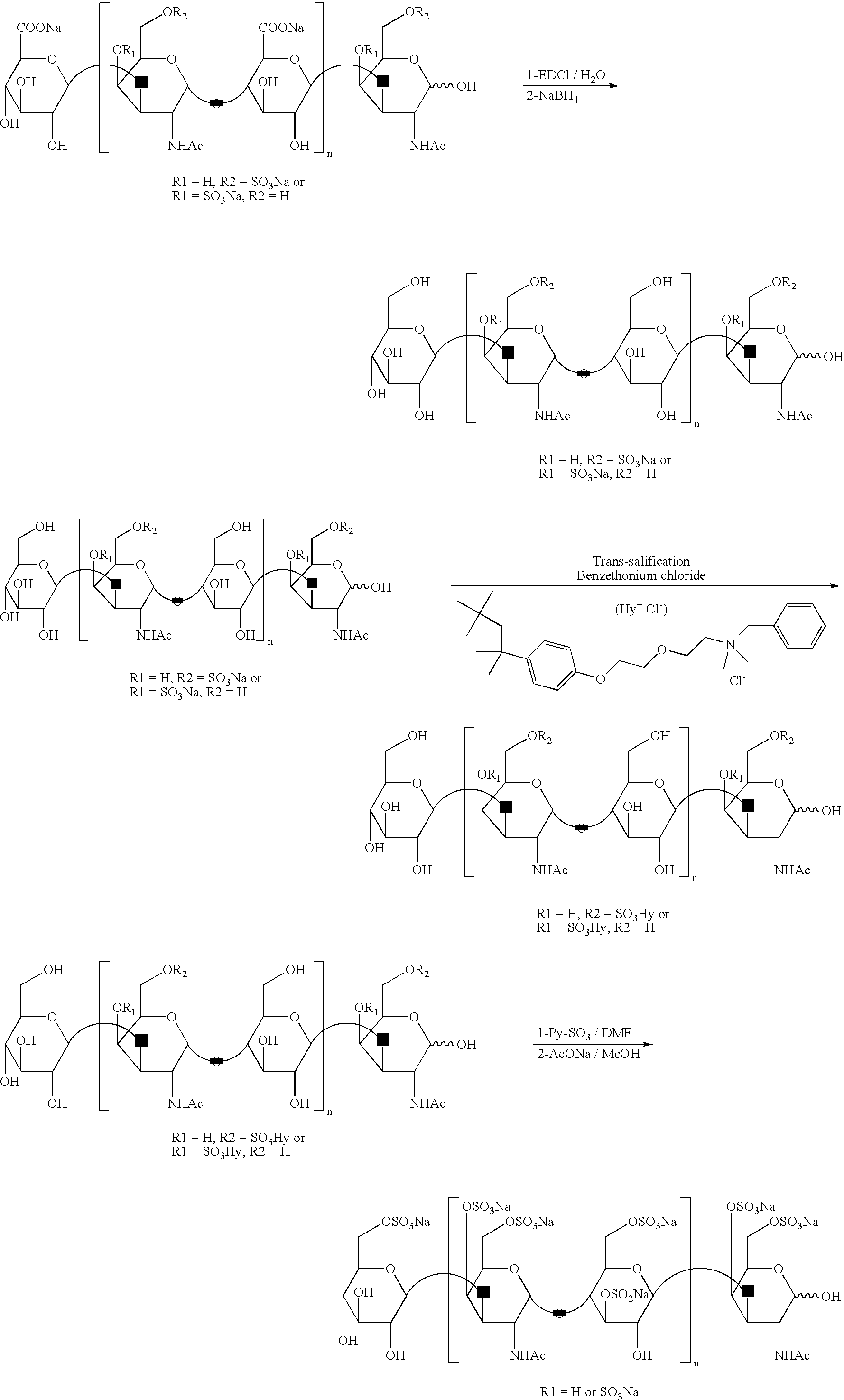

a) Preparation of the Carboxyl-Reduced Chondroitin Sulfate Sodium Salt.

The carboxyl-reduced chondroitin sulfate sodium salt is prepared according to Example 1-a.

b) Preparation of the Carboxyl-Reduced Chondroitin Sulfate Benzethonium Salt.

The carboxyl-reduced chondroitin sulfate benzethonium salt is prepared according to example 1-b.

c) Preparation of the Carboxyl-Reduced and 6,6′-O-sulfated Chondroitin Sulfate Sodium Salt

0.13 g of carboxyl-reduced chondroitin sulfate benzethonium salt is dissolved, at a temperature in the region of 20° C. and under an inert atmosphere, in 10 ml of anhydrous dimethylformamide. When dissolution is complete, a solution of 0.2 g of pyridine-sulfuric anhydride complex in 5 ml of anhydrous dimethylformamide is added to the above solution, at a temperature in the region of −10° C. After stirring for 2 hours 30 min at a temperature in the region of −10° C., 2 ml of water and then 60 ml of a 10% sodium acetate solution in methanol are added. The suspension obtained is stirred for 30 minutes, left to sediment overnight, and then filtered. The cake is washed with 5 ml of methanol, dissolved in 20 ml of water, and then precipitated by adding 60 ml of a 10% sodium acetate solution in methanol. The suspension obtained is filtered. The cake is washed with methanol (twice 5 ml) and then dissolved in 10 ml of water. The solution obtained is loaded into a PM 3500 cellulose ester membrane and dialyzed, changing the water baths regularly for 5 days. The content of the membrane is lyophilized to give 0.074 g of carboxyl-reduced and 6,6′-O-sulfated chondroitin sulfate sodium salt in the form of a white lyophilized material. The yield obtained is 83%.

Proton spectrum in D₂O, 400 MHz, T=333 K (δ in ppm—mixture of 4 chondroitins, possibly sulfated in the C4 position (Gal 2N6S), possibly epimerized in the C5 position (Glu6S)): 2.0 (3H, s), 3.30 (1H, m), between 3.5 and 4.25 (11H, m), between 4.4 and 4.7 (2H, m), between 4.75 and 4.92 (1/2H).

### EXAMPLE 3

### Preparation of the Persulfated, Carboxyl-Reduced Chondroitin Sulfate Sodium Salt (Compounds of Formula (I) with the Groups R₁═R₂═R₄═SO₃Na and R₃═H)

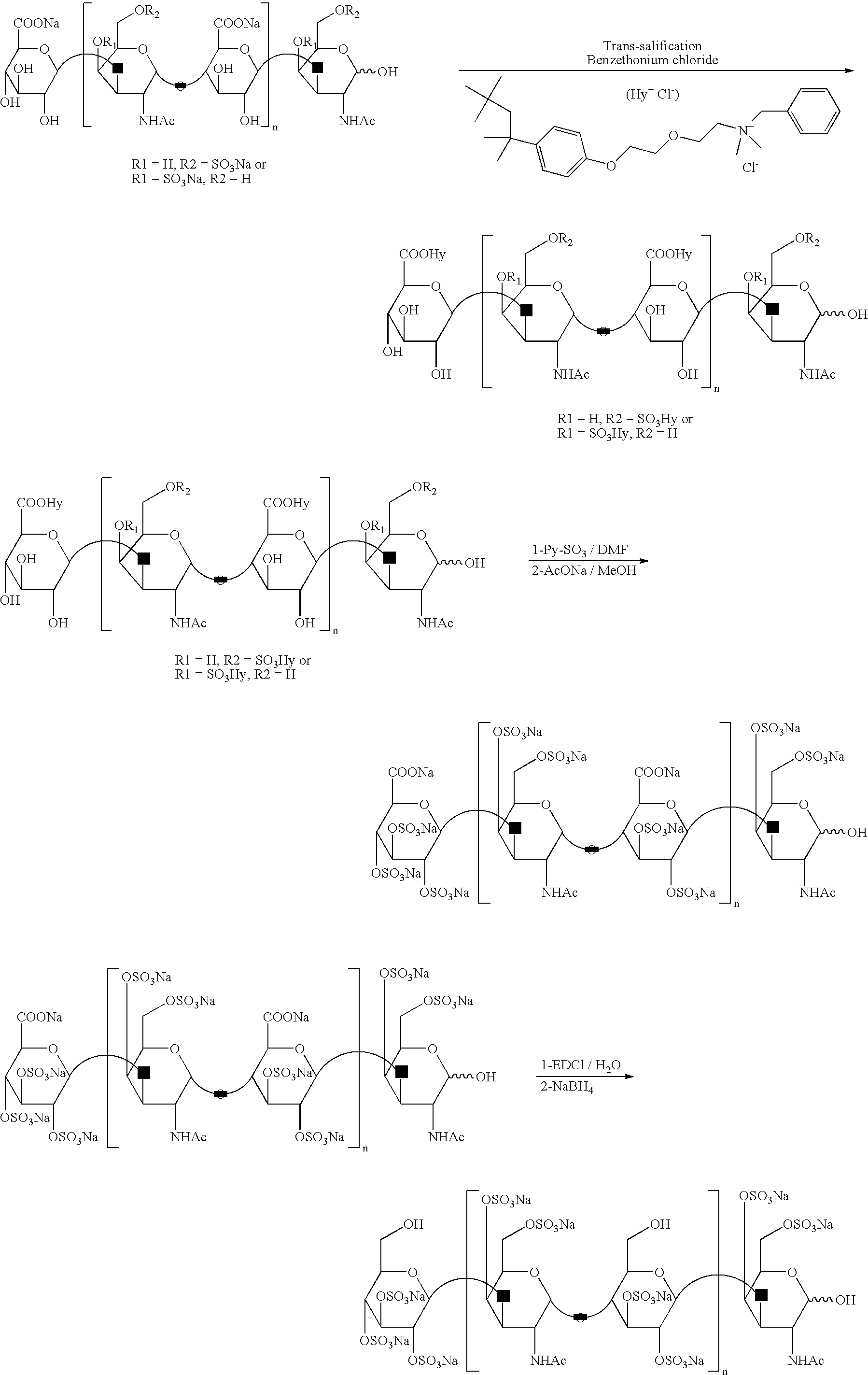

a) Preparation of the Chondroitin Sulfate Benzethonium Salt.

A solution of 113 g of benzethonium chloride in 560 ml of water is added to the solution of 50 g of chondroitin sulfate sodium salt in 715 ml of water. The suspension obtained is stirred for 1 hour at a temperature in the region of 20° C. and then left to sediment overnight. The supernatant is removed by settling out and then replaced with the same volume of water. The suspension is again stirred for one hour and then left to sediment. The operation described above is repeated 3 times. The suspension is then filtered. The cake is washed with water and then dried at 40° C. under reduced pressure (6 kPa) to give 123.4 g of chondroitin sulfate benzethonium salt in the form of a white solid. The yield obtained is 94%.

b) Preparation of the Persulfated Chondroitin Sulfate Sodium Salt

5 g of chondroitin sulfate benzethonium salt are dissolved, at a temperature in the region of 60° C. and under an inert atmosphere, in 100 ml of anhydrous dimethylformamide. When dissolution is complete, a solution of 26.5 g of pyridine-sulfuric anhydride complex in 250 ml of anhydrous dimethylformamide is added to the above solution. After stirring for 1 hour 30 min at a temperature in the region of 60° C., the reaction medium is cooled to a temperature in the region of 10° C. 100 ml of water and then 1350 ml of a 10% sodium acetate solution in methanol are added. The suspension obtained is filtered. The cake is washed with methanol (50 ml), dissolved in 400 ml of water, and then precipitated by adding 1200 ml of a 10% sodium acetate solution in methanol. The suspension obtained is filtered. The cake is washed with methanol (20 ml) and then dissolved in 200 ml of water. The solution obtained is loaded into a PM 3500 cellulose ester membrane and dialyzed, changing the water baths regularly for 4 days. The content of the membrane is lyophilized to give 2 g of persulfated chondroitin sulfate sodium salt in the form of a white lyophilized material. The yield obtained is 66%.

Proton spectrum in D₂O, 400 MHz, T=298 K (δ in ppm—mixture of chondroitins A and C): 2.05 (3H, s), 4.0 (3H, m), 4.1 (1H, s), 4.23 (2H, m), 4.4 (2H, m), 4.7 (1H, m), 4.82 (1H, d, J=3 Hz), 4.87 (1H, s), 4.95 (1H, s).

c) Preparation of the Persulfated, Carboxyl-Reduced Chondroitin Sulfate Sodium Salt

0.5 g of persulfated chondroitin sulfate sodium salt are dissolved, at a temperature in the region of 20° C., in 45 ml of water. The pH of the solution is adjusted to a value of 4.7±0.1 by adding a 0.1 N hydrochloric acid solution. 1.1 g of 1-ethyl-3-(3-dimethylamino-propyl)carbodiimide hydrochloride are added and the mixture obtained is stirred while maintaining the pH at a value of 4.7±0.1 by adding a 0.1 N hydrochloric acid solution. When the pH is stabilized at a value in the region of 4.7, 4.44 g of sodium borohydride are added in small portions. The reaction medium is stirred for approximately 20 hours at a temperature in the region of 20° C. After cooling to a temperature in the region of 10° C., the reaction medium is neutralized by adding concentrated hydrochloric acid (10 N) to a pH in the region of 7. The reaction medium is loaded into a PM 3500 cellulose ester membrane and dialyzed, changing the water baths regularly for 65 hours. 600 ml of a 10% sodium acetate solution in methanol are added to the content of the membranes. The solid obtained is filtered, and then dissolved in 200 ml of water. The solution obtained is loaded into a PM 3500 cellulose ester membrane and dialyzed, changing the water baths regularly for 28 hours. The content of the membranes is lyophilized to give 0.28 g of persulfated, carboxyl-reduced chondroitin sulfate sodium salt in the form of a white lyophilized material. The yield obtained is 59%.

Proton spectrum in D₂O, 400 MHz, T=323 K (δ in ppm—mixture of chondroitins A and C): 2.0 (3H, s), 3.65 (3H, m), 3.89 (3H, m), 3.96 (1H, m), 4.05 (1H, s), 4.2 (2H, s), 4.7 (2H, m), 4.89 (1H, s), 4.93 (1H, s).

### EXAMPLE 4

### Preparation of the 6-O-sulfated, Carboxyl-Reduced Chondroitin Sulfate Sodium Salt (Compounds of Formula (I) with the Groups R₁═H/SO₃Na R₂═SO₃Na and R₃═R₄═H)

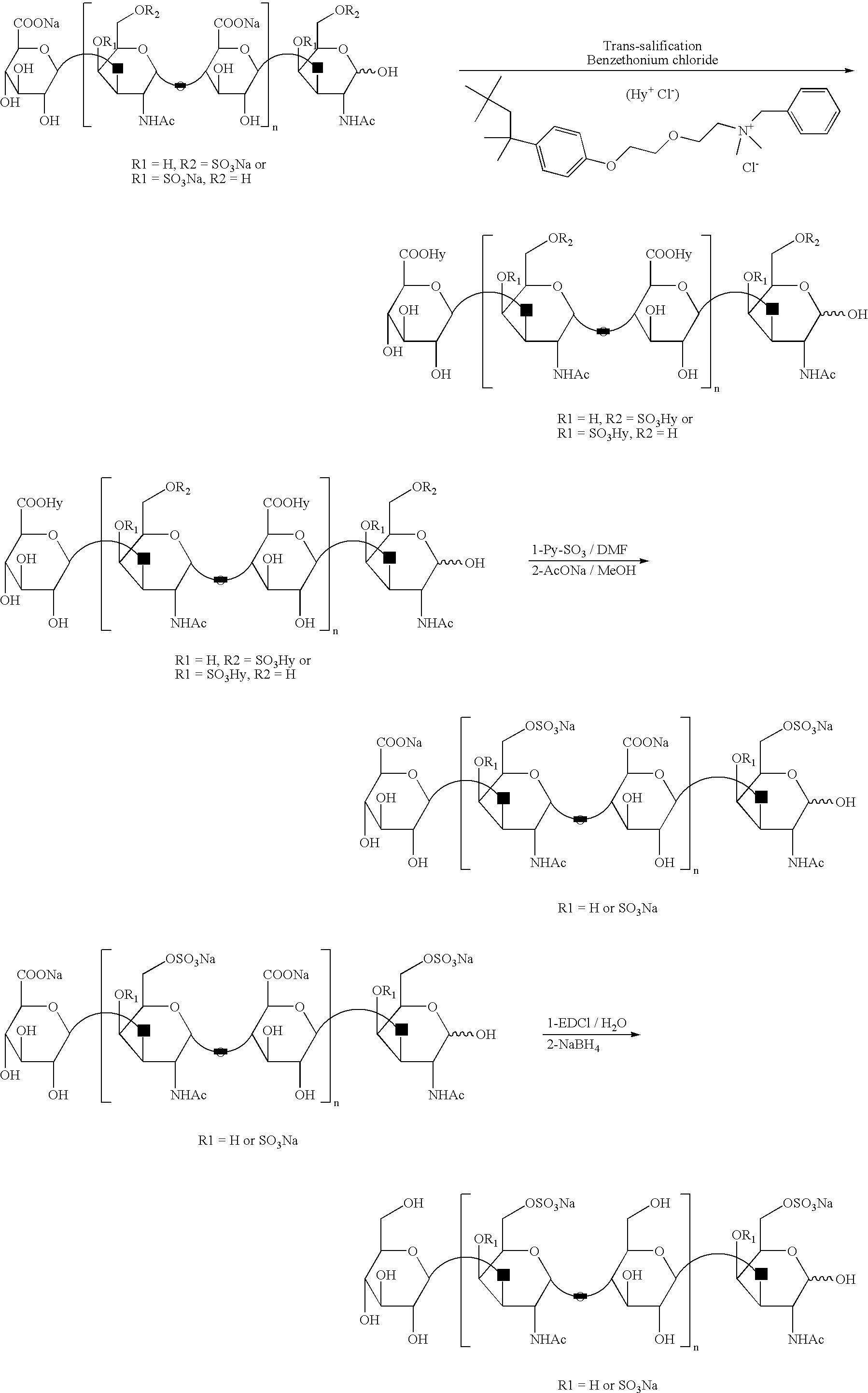

a) Preparation of the Chondroitin Sulfate Benzethonium Salt

The chondroitin sulfate benzethonium salt is prepared according to example 3-a.

b) Preparation of the 6-O-sulfated Chondroitin Sulfate Sodium Salt

10 g of chondroitin sulfate benzethonium salt are dissolved, at a temperature in the region of 0° C. and under an inert atmosphere, in 200 ml of anhydrous dimethylformamide. When dissolution is complete, a solution of 10.6 g of pyridine-sulfuric anhydride complex in 100 ml of anhydrous dimethylformamide is added to the above solution. After stirring for 2 hours 30 min at a temperature in the region of 0° C., 150 ml of water and then 1600 ml of a 10% sodium acetate solution in methanol are added. The suspension obtained is filtered. The cake is dissolved in 500 ml of water and then precipitated by adding 1500 ml of a 10% sodium acetate solution in methanol. The suspension obtained is filtered. The cake is washed with methanol (20 ml), and then dissolved in 200 ml of water. The solution obtained is loaded into a PM 3500 cellulose ester membrane and dialyzed, changing the water baths regularly for 24 hours. The content of the membrane is lyophilized to give 2.89 g of 6-O-sulfated chondroitin sulfate sodium salt in the form of a white lyophilized material. The yield obtained is 69%.

Proton spectrum in D₂O, 400 MHz, T=323 K (δ in ppm—mixture of chondroitins A and C): 2.0 (3H, s), 3.35 (1H, m), 3.55 (1H, m), between 3.62 and 4.38 (3H+1/2H, m), between 3.4 and 4.1 (2H, m), 4.2 (2H, m), 4.45 (1H, m), 4.55 (1H, m), 4.78 (1/2H, s).

c) Preparation of the 6-O-sulfated, Carboxyl-Reduced Chondroitin Sulfate Sodium Salt

2 g of 6-O-sulfated chondroitin sulfate sodium salt are dissolved, at a temperature in the region of 20° C., in 250 ml of water. The pH of the solution is adjusted to a value of 4.7±0.1 by adding a 0.1 N hydrochloric acid solution. 6.5 g of 1-ethyl-3-(3-dimethylaminopropyl)-carbodiimide hydrochloride are added and the mixture obtained is stirred while maintaining the pH at a value of 4.7±0.1 by adding a 0.1 N hydrochloric acid solution. When the pH is stabilized at a value in the region of 4.7, 25.8 g of sodium borohydride are added in small portions. The reaction medium is stirred for approximately 20 hours at a temperature in the region of 20° C. After cooling to a temperature in the region of 10° C., the reaction medium is neutralized by adding concentrated hydrochloric acid (10 N) to a pH in the region of 7. The reaction medium is loaded into a PM 3500 cellulose ester membrane and dialyzed, changing the water baths regularly for 50 hours. 1530 ml of a 10% sodium acetate solution in methanol are added to the content of the membranes. The solid obtained is filtered, washed with methanol, and then dissolved in 100 ml of water. The solution obtained is loaded into a PM 3500 cellulose ester membrane and dialyzed, changing the water baths regularly for 24 hours. The content of the membranes is lyophilized to give 0.758 g of 6-O-sulfated, carboxyl-reduced chondroitin sulfate sodium salt in the form of a white lyophilized material. The yield obtained is 41%.

Proton spectrum in D₂O, 400 MHz, T=298 K (δ in ppm—mixture of chondroitins A and C (1/3 4-OSO₃H, 2/3 6-OSO₃H)): 2.0 (3H, s), 3.30 (1H, m), between 3.4 and 3.90 (7H+1/3H, m), between 3.92 and 4.05 (2H, m), 4.19 (4/3H, s), between 4.4 and 4.6 (2H, m), 4.88 (1/3H, s).

### EXAMPLE 5

### Preparation of the Carboxyl-Reduced and Persulfated Chondroitin Sulfate Sodium Salt (Compounds of Formula (I) with the Groups R₁═R₂═R₃═R₄═SO₃Na)

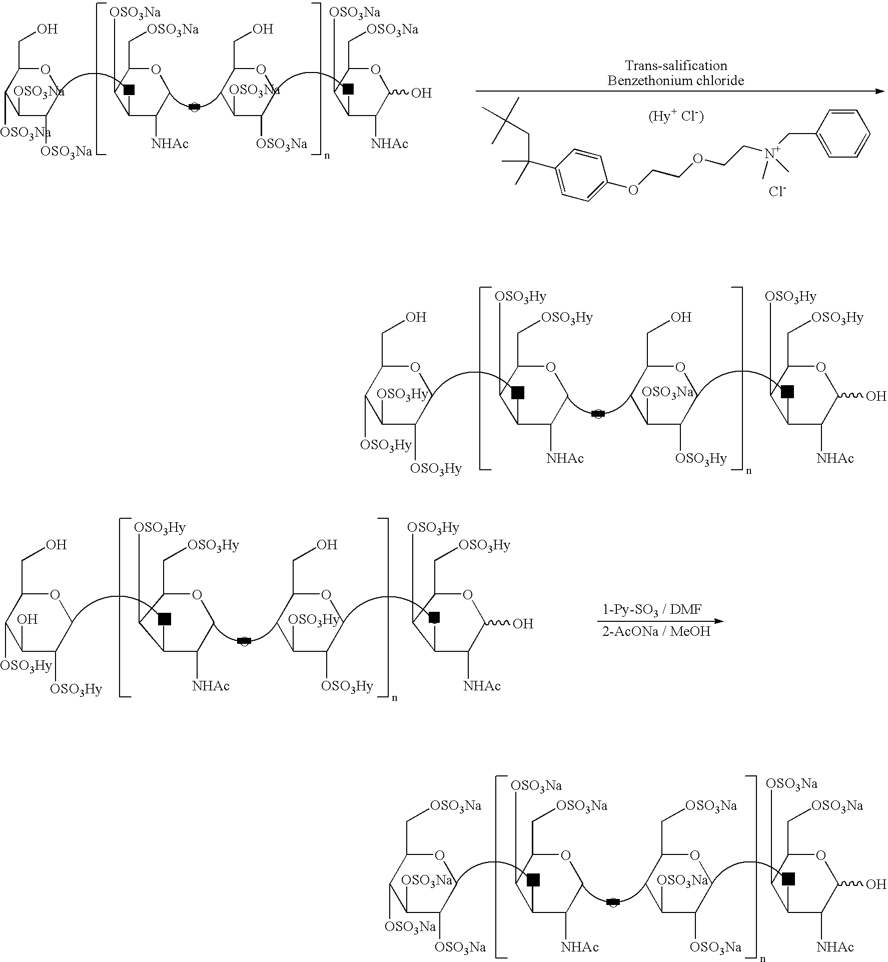

a) Preparation of the Persulfated, Carboxyl-Reduced Chondroitin Sulfate Benzethonium Salt

A solution of 0.9 g of benzethonium chloride in 15 ml of water is added to the solution of 0.4 g of persulfated, carboxyl-reduced chondroitin sulfate sodium salt (prepared according to example 3) in 15 ml of water. The suspension obtained is stirred for 1 hour at a temperature in the region of 20° C., left to sediment overnight, and then filtered. The cake is washed with water (6 times 50 ml), dried in a dessicator under vacuum, and then at 80° C. under reduced pressure (6 kPa), to give 0.95 g of persulfated, carboxyl-reduced chondroitin sulfate benzethonium salt in the form of a white solid. The yield obtained is 80%.

b) Preparation of the Carboxyl-Reduced and Persulfated Chondroitin Sulfate Sodium Salt

0.95 g of persulfated, carboxyl-reduced chondroitin sulfate benzethonium salt are dissolved, at a temperature in the region of 20° C. and under an inert atmosphere, in 10 ml of anhydrous dimethylformamide. When dissolution is complete, a solution of 1.27 g of pyridine-sulfuric anhydride complex in 5 ml of anhydrous dimethylformamide is added to the above solution. After stirring for 2 hours 30 min at a temperature in the region of 20° C., the reaction medium is cooled to a temperature in the region of 10° C. 7 ml of water and then 60 ml of a 10% sodium acetate solution in methanol are added. The suspension obtained is filtered. The cake is dissolved in 50 ml of water. The suspension obtained is filtered. The filtrate is precipitated by adding 120 ml of a 10% sodium acetate solution in methanol. The suspension obtained is filtered. The cake is dissolved in 10 ml of water. The solution obtained is loaded into a PM 3500 cellulose ester membrane and dialyzed, changing the water baths regularly for 26 hours. The content of the membrane is lyophilized to give 0.17 g of carboxyl-reduced and persulfated chondroitin sulfate sodium salt in the form of a white lyophilized material. The yield obtained is 48%.

Proton spectrum in D₂O, 400 MHz, T=298 K (δ in ppm—mixture of chondroitins A and C): 2.05 (3H, s), 3.85 (1H, m), between 3.92 and 4.05 (3H, m), between 4.12 and 4.3 (5H, m), 4.58 (1H, s), 4.65 (1H, s), 4.75 (1H, m), 4.95 (1H, s), 5.0 (1H, s).

### EXAMPLE 6

### Preparation of the Carboxyl-Reduced and 6,6′-O-sulfated Chondroitin Sulfate Sodium Salt (Compounds of Formula (I) with the Groups R₁═H/SO₃Na R₂═R₃═SO₃Na and R₄═H)

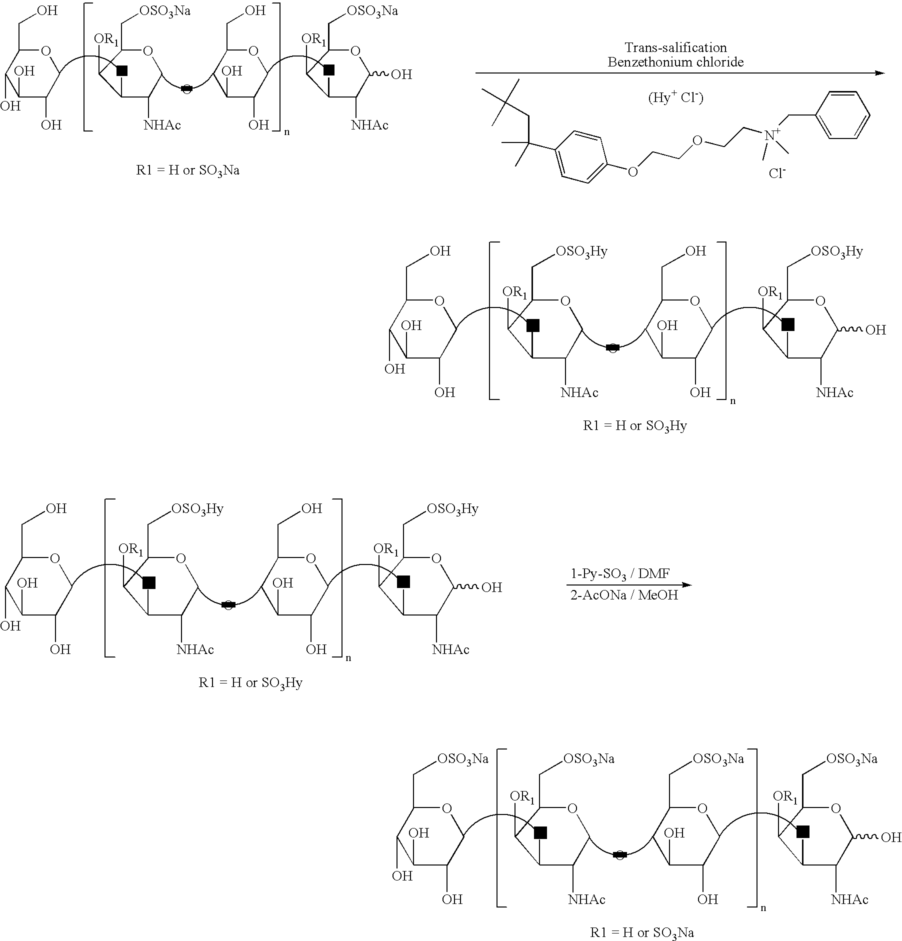

a) Preparation of the 6-O-sulfated, Carboxyl-Reduced Chondroitin Sulfate Benzethonium Salt

A solution of 1.9 g of benzethonium chloride in 30 ml of water is added to the solution of 0.8 g of 6-O-sulfated, carboxyl-reduced chondroitin sulfate sodium salt (prepared according to example 4) in 30 ml of water. The suspension obtained is stirred for 1 hour at a temperature in the region of 20° C., left to sediment overnight, and then filtered. The cake is washed with water (5 times 25 ml), dried in a desiccator under vacuum, and then at 60° C. under reduced pressure (6 kPa), to give 1.64 g of 6-O-sulfated, carboxyl-reduced chondroitin sulfate benzethonium salt in the form of a white solid. The yield obtained is 96%.

b) Preparation of the Carboxyl-Reduced and 6,6′-O-sulfated Chondroitin Sulfate Sodium Salt

0.5 g of 6-O-sulfated, carboxyl-reduced chondroitin sulfate benzethonium salt is dissolved, at a temperature in the region of 20° C. and under an inert atmosphere, in 8 ml of anhydrous dimethylformamide. When dissolution is complete, a solution of 0.65 g of pyridine-sulfuric anhydride complex in 4 ml of anhydrous dimethylformamide is added to the above solution, at a temperature in the region of −10° C. The reaction medium is stirred at a temperature in the region of −10° C. for 2 hours 30 min. 5 ml of water and then 80 ml of a 10% sodium acetate solution in methanol are added. The suspension obtained is filtered. The cake is washed with methanol, dissolved in 20 ml of water, and then precipitated by adding 60 ml of a 10% sodium acetate solution in methanol. The suspension obtained is filtered. The cake is dissolved in 10 ml of water. The solution obtained is loaded into a PM 3500 cellulose ester membrane and dialyzed, changing the water baths regularly for 26 hours. The content of the membrane is lyophilized to give 0.22 g of carboxyl-reduced and 6,6′-O-sulfated chondroitin sulfate sodium salt in the form of a white lyophilized material. The yield obtained is 79%.

Proton spectrum in D₂O, 400 MHz, T=333 K (δ in ppm—mixture of 4 chondroitins, possibly sulfated in the C4 position (Gal 2N6S), possibly epimerized in the C5 position (Glu6S)): 2.0 (3H, s), 3.30 (1H, m), between 3.5 and 4.25 (11H, m), between 4.4 and 4.7 (2H, m), between 4.75 and 4.92 (1/2H).

Pharmacological Test

Effect of the carboxyl-reduced derivatives on the aggrecanase in synovial fluids or on preparations of recombinant ADAMTS protein.

The analysis is carried out on 96-well plates. Before the analysis, serial dilutions of the assay compounds are prepared in an aqueous solution.

Digestion:

In each well, a fixed volume of synovial fluid or aggrecanase activity generating a known increase, between 1.0 and 1.4 absorbance units (405 nm), under the conditions of the analysis, is mixed with 3 μl of solution of compound from the respective dilution step. Dulbecco's modified Eagle's medium (DMEM) is added to each well to give a final volume of 300 μl. The plate is then incubated for 1 hour at 37° C. in a carbon dioxide atmosphere.

After the addition of 5 μl of a solution of 1 μg/μl of recombinant substrate Agglmut in DMEM to each well (substrate as described by Bartnik E. et al., EP 785274, 1997), the reagent mixture is incubated for 4 hours at 37° C. under a carbon dioxide atmosphere.

Preparation of the Analytical Plate:

In a first step, the microplate is coated with 100 μl per well of a commercial goat anti-mouse IgG antibody for 1 hour at ambient temperature (5 μg/ml in a phosphate buffered saline solution, pH 7.4 [PBS buffer]). After washing with the PBS buffer containing 0.1% of Tween-20 (washing buffer), the wells are blocked by means of a step consisting of a one hour incubation with 100 μl per well of 5% bovine serum albumin in a PBS buffer containing 0.05% of Tween-20. Following another rinse with the washing buffer, each well is incubated with 100 μl of a solution, diluted to 1:1000, of BC-3 antibody in a PBS buffer containing 0.05% of Tween 20 and 0.5% of bovine serum albumin, at ambient temperature for one hour. This antibody recognizes the typical aggrecanase cleavage fragments (Hughes C. E. et al., Biochem. J. 305 (3), 799-804, 1995).

Assay Procedure:

After another rinse of the analytical plate with the washing buffer, the complete set of mixtures originating from the preceding digestion is transferred to this plate, well by well, and incubated at ambient temperature for one hour. The plate is again washed as above. Next, 100 μl of the second antibody (anti-human IgG antibody labeled with peroxidase, 1:1000 in PBS containing 0.5% of BSA and 0.05% of Tween-20) are added to each well, with subsequent incubation at ambient temperature again for one hour. After a final rinse with the washing buffer, the color development is initiated by adding 100 μl of ABTS substrate solution (2 mg/ml, 2,2′-azinobis (3-ethylbenzothiazolinesulfonic acid) in 40 mM of sodium citrate plus 60 mM of disodium hydrogen phosphate, adjusted to a pH of 4.4 by adding acetic acid; 0.25 μl of 35% hydrogen peroxide added per ml immediately before measurement). The measurement is carried out by means of the screen mode using detection at 405 nm relative to a reference filter (620 nm) with automatic readings at 5-second intervals. The development is stopped as soon as the maximum signal (405 nm) in the absorbance range of 1.0 to 1.4 is reached.

Table 1

% inhibition of conversion of the substrate by activity of the aggrecanase in the supernatant.

| Concentration | |
|---|---|
| of glycosaminoglycan | |
| (μg/ml) | % Conversion |
| 0.03 | 14.9 |
| 0.01 | 11.6 |
| 0.003 | 69.2 |
| 0.001 | 95.1 |
| 0.0003 | 99.0 |
| 0.0001 | 100 |

The estimated IC₅₀ of the product of example 1 is of the order of 0.004 μg/ml
Inhibition of Factor Xa

For the calibration, a standard sample of low molecular weight heparin (Enoxaparin) was used as reference.

The serial dilutions of the assay compound are prepared in a 0.046 M Tris buffer, pH 8.4, containing 0.15 M NaCl, 0.007 M EDTA, 0.1% of Tween 80 and 0.12 IU/ml of human antithrombin III. The samples of 50 μl of the respective dilutions are incubated with 50 μl of bovine factor Xa (13.6 U/ml) at 37° C. for 80 seconds. Next, 50 μl of chromogenic substrate, 1.1 mM S-2765, are added. The absorbance at 405 nm is measured in a photometer. The activity of the deblocked factor Xa is indicated by the emission of p-nitroaniline from the substrate.

Compared to the reference heparin compound (100 U/mg), the glycosamonoglycans present a factor Xa, with much lower inhibitory activity:

### EXAMPLE 1

### 1.2 U/mg

### Effect of the Carboxyl-Reduced Persulfate Derivative (Example 1) for Human Metalloproteases MMP-2 (gelatinase-A) and MMP7

Commercial ELISA kits (such as those provided, for example, by the company Amersham Biosciences, kits RPN2617 and RPN2620) were used, respectively, to determine the inhibitory activity with respect to the MMP-2 and MMP-7 enzymes. The concentrations of the enzymes are, respectively, 800 ng/ml for MMP-2. The tests were carried out in accordance with the manufacturer's recommendations, by performing a series of dilutions of the compound to be studied in a PBS buffer, pH 7.5. The MMP-2 enzymes were inhibited in a concentration-dependent manner.

Estimated IC₅₀: 0.4 μg/ml

1. A carboxyl-reduced and chemoselectively O-sulfated compound of chondroitin sulfate, isolated as a single compound or as a mixture, or the salts thereof.

2. A carboxyl-reduced compound of chondroitin sulfate as claimed in claim 1, of formula (I): 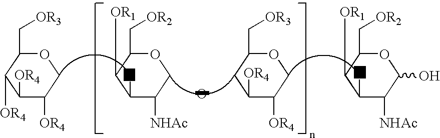
in which R₁, R₂, R₃ and R₄, which may be identical or different, are H or SO₃M, it being understood that at least one of the substituents R₁, R₂, R₃ and R₄ represents a group SO₃M, n is an integer of between 0 and 200, M is an alkali metal, said compound isolated as a single compound or a mixture, or the diastereoisomers thereof.

3. A carboxyl-reduced compound of chondroitin sulfate as claimed in claim 2, wherein M is chosen from sodium, calcium, magnesium and potassium.

4. A carboxyl-reduced compound of chondroitin sulfate as claimed in claim 3, wherein M is a sodium atom and n is an integer of between 100 and 200.

5. A carboxyl-reduced compound of chondroitin sulfate as claimed in claim 2, wherein R₁, R₂, R₃ and R₄ are SO₃Na.

6. A carboxyl-reduced compound of chondroitin sulfate as claimed in claim 2, wherein R₁, R₂ and R₄ are SO₃Na and R₃ is H.

7. A carboxyl-reduced compound of chondroitin sulfate as claimed in any one of claims 2, wherein R₁ is H or SO₃Na, R₂ is SO₃Na and R₃ and R₄ are H.

8. A carboxyl-reduced compound of chondroitin sulfate as claimed in any one of claims 2, wherein R₁ is H or SO₃Na, R₂ and R₃ are SO₃Na and R₄ is H.

9. A process for preparing the carboxyl-reduced compounds of chondroitin sulfate as claimed in claim 1, comprising the following steps:
reducing the carboxyl group of chondroitin sulfate
optionally a) reducing a carbodiimide compound (forming an activated adduct) in the presence of a reducing agent,
or b) by esterifying, the chondroitin sulfate having, where appropriate, been trans-salified beforehand with a quaternary ammonium salt, followed by reducing the corresponding ester compound by the action of a reducing agent,
where appropriate, trans-salifying of the carboxyl-reduced compound with a quaternary ammonium salt; and
sulfating in organic medium, followed by salifying.

10. A process for preparing the carboxyl-reduced compounds of chondroitin sulfate as claimed in claim 1, comprising the following steps:
trans-salifying of chondroitin sulfate with a quaternary ammonium salt,
sulfating in organic medium, followed by salifying,
reducing the carboxyl group of the sulfated chondroitin sulfate
optionally a) by means of a carbodiimide compound (forming an activated adduct) in the presence of a reducing agent,
or b) by esterifying of chondroitin sulfate, wherein said chondroitin sulfate having, where appropriate, been trans-salified beforehand with a quaternary ammonium salt, followed by reducing the corresponding ester compound by the action of a reducing agent; and
where appropriate, trans-salifying the persulfated, carboxyl-reduced compound with a quaternary ammonium salt, and then resulfating followed by salifying.

11. The process as claimed in claim 9, wherein the sulfating is carried out in organic medium by means of a complex of sulfuric anhydride with an organic base chosen from pyridine and trimethylamine, at a temperature of between about −15° C. and about 70° C.

12. The process as claimed in claim 9, wherein the the carboxyl-reduced chondroitin sulfate quaternary ammonium salt to be sulfated is a benzethonium salt compound of formula (I) wherein R₁ is H, R₂ is SO₃Na or R₁ is SO₃Na, R₂ is H and R₃ and R₄ and are H, and wherein the sulfating of said salt is carried out in the presence of about 10 to about 30 equivalents of pyridine-sulfuric anhydride complex per hydroxyl function to be sulfated, and at a temperature of between about 50° C. and about 70° C., in order to obtain a compound of formula (I) as defined in claim 5 (R₁,R₂,R₃ and R₄ are SO₃Na).

13. The process as claimed in claim 9, wherein the carboxyl-reduced chodroitin sulfate quaternary ammonium salt to be sulfated is a benzethonium salt as defined in claim 6, compound of formula (I) wherein R₁,R₂ and R₄ are SO₃Na and R₃ is H, and wherein the sulfating of said salt is carried out in the presence of about 10 to about 30 equivalents of pyridine-sulfuric anhydride complex, at about 20° C., in order to obtain a compound of formula (I) as defined in claim 5 (R₁,R₂,R₃ and R₄ are SO₃Na).

14. The process as claimed in claim 9, wherein the carboxyl-reduced chodroitin sulfate quaternary ammonium salt to be sulfated is a benzethonium salt, compound of formula (I) wherein R₁ is H, R₂ is SO₃Na or R₁ is SO₃Na, R₂ is H and R₃ and R₄ are H, and wherein the sulfating of said salt is carried out in the presence of about 5 to about 15 equivalents of pyridine-sulfuric anhydride complex, at temperatures of between about −15° C. and about 5° C., in order to obtain a compound of formula (I) as defined in claim 8 wherein R₁ is H, or SO₃Na, R₂ and R₃ are SO₃Na and R₄ is H.

15. The process as claimed in claim 10, wherein the resulfating is carried out using as the quaternary ammonium salt, carboxyl-reduced, 6-O-sulfated chondroitin sulfate benzethonium salt, compound of formula (I) wherein R₁ is H or SO₃Na, R₂ is SO₃Na and R₃ and R₄ are H) as defined in claim 7, in the presence of about 5 to about 15 equivalents of pyridine-sulfuric anhydride complex, at temperatures of between about −15° C. and about 5° C., and in the presence of about 10 equivalents of pyridine-sulfuric anhydride complex, at about −10° C., in order to obtain compounds of formula (I) wherein R₁ is H or SO₃Na R₂ and R₃ are SO₃Na and R₄ is H as defined in claim 8.

16. The process as claimed in claim 9, further comprising reducing the carboxyl group in the presence of 1-(3dimethylaminopropyl)-3-ethylcarbodiimide hydrochloride and an alkali metal borohydride.

17. The process as claimed in claim 16, wherein between about 5 to about 20 equivalents of 1-(3-dimethylaminopropyl)-3-ethylcarbodiimide hydrochloride are used, and at a pH of between about 4 and about 5.

18. The process as claimed in claim 17, wherein the pH is between about 4.3 to about 4.9

19. The process as claimed in claim 9, further comprising reducing the activated adduct of the chondroitin sulfate compound with about 10 to about 300 equivalents of alkali metal borohydride at a temperature of between about 10° C. and about 70° C.

20. The process as claimed in claim 19, wherein the temperature range is between about 10° C. and about 30° C.

21. The process as claimed in claim 19, wherein the alkali metal borohydride is sodium borohydride.

22. The process as claimed in claim 9, wherein the ester to be reduced is a methyl ester and said reducing agent is an alkali metal borohydride.

23. The process as claimed in claim 22 wherein the alkali metal hydride is sodium borohydride.

24. The process as claimed in claim 9, comprising the following steps:
optionally a) reducing the carboxyl groupo of chondroitin sulfate sodium salt in the presence of 1-(3-dimethylaminopropyl)-3-ethylcarbodiimide hydrochloride sodium borohydride,
or b) esterifying by the action of methyl iodide on the chondroitin sulfate compound trans-salified beforehand with benzethonium chloride, and then reducing the corresponding methyl ester compound with sodium borohydride, then
trans-salifying of the carboxyl-reduced compound with benzethonium chloride,
sulfating of the chondroitin sulfate benzethonium salt in organic medium by means of a complex of sulfuric anhydride with an organic base chosen from pyridine and trimethylamine, followed by salifying with sodium acetate.

25. The process as claimed in claim 10, wherein the sulfating is carried out in organic medium by means of a complex of sulfuric anhydride with an organic base chosen from pyridine and trimethylamine, at a temperature of between about −15 and about 70° C.

26. The process as claimed in claim 10, further comprising reducing the carboxyl group in the presence of 1-(3-dimethylaminopropyl)-3-ethylcarbodiimide hydrochloride and an alkali metal borohydride.

27. The process as claimed in claim 26, wherein between about 5 to about 20 equivalents of 1-(3-dimethylaminopropyl)-3-ethylcarbodiimide hydrochloride are used, and at a pH of between about 4 and about 5.

28. The process as claimed in claim 27, wherein the pH is between about 4.3 to about 4.9

29. The process as claimed in claim 10, further comprising reducing the activated adduct of the chondroitin sulfate compound with about 10 to about 300 equivalents of alkali metal borohydride at a temperature of between 10 and 70° C.

30. The process as claimed in claim 30, wherein the temperature range is between about 10° C. and about 30° C.

31. The process as claimed in claim 29 wherein the alkali metal hydride is sodium borohydride.

32. The process as claimed in claim 10, wherein the ester to be reduced is a methyl ester and said reducing agent is an alkali metal borohydride.

33. The process as claimed in claim 32, wherein the alkali metal hydride is sodium borohydride.

34. The process as claimed in claim 10, further comprising the following steps:
trans-salifying of the chondroitin sulfate sodium salt with benzethonium chloride,
sulfating of the chondroitin sulfate benzethonium salt in organic medium by means of a complex of sulfuric anhydride with an organic base chosen from pyridine and trimethylamine, followed by salifying with sodium acetate,—optionally a) reducing the carboxyl group of chondroitin sulfate in the presence of 1-(3-dimethylaminopropyl)-3-ethylcarbodiimide hydrochloride and sodium borohydride,
or b) esterifying by reacting methyl iodide with chondroitin sulfate trans-salified beforehand with benzethonium chloride, and then reduction of the corresponding methyl ester compound with sodium borohydride; and
where appropriate, trans-salifying of the persulfated, carboxyl-reduced compound with benzethonium chloride, then resulfating with the sulfuric anhydride-organic base complex, followed by salifying with sodium acetate.

35. A process for obtaining the isolated carboxyl-reduced and chemoselectively O-sulfated compounds of chondroitin sulfate as claimed in claims 1, from the mixture of carboxyl-reduced compounds of chondroitin sulfate further comprising fractionating said mixture by column chromatography on columns filled with polyacrylamide agarose gel or a polyacrylamide gel, said mixture being eluted with a sodium hydrogen carbonate solution, in a concentration of about 0.1 to about 1 mol/liter.

36. A pharmaceutical composition comprising a compound of claim 1 and one or more pharmaceutically acceptable excipients.

37. A method of treating a disease in a patient characterized by an increased activity in at least one of the matrix metalloproteinases selected from the group consisting of collagenase, elastase matrisylin (MMP-7), aggrencanase, hADAMTS1 and gelatinase A (MMP-2), comprising administering to said patient a therapeutically effective amount of a compound as claimed in claim 1.

38. The method of claim 37 wherein the disease is selected form the group consisting of joint degeneration, spondylosis, chondrolysis associated with joint trauma or prolonged immobilization of the joint, connective tissue disorders, wound healing conditions, periodontal disorders, chronic disorders of the locomotor system, arthropathies, myalgias and bone metabolism disorders.
